(19) United States
(12) Patent Application Publication
(10) Pub. No.: US 2003/0027856 A1
(43) Pub. Date: Feb. 6, 2003

(54) Tolterodine metabolites

(76) Inventor: A.K. Gunnar Aberg, Sarasota, FL (US)

Correspondence Address: Kevin S. Lemack, Nields & Lemack, Suite 8, 176 E. Main Street, Westboro, MA, 01581, US

(21) Appl. No.: 09/895,463

(22) Filed: Jun. 29, 2001

(51) Int. Cl. A01N 43/16; A61K 31/35; C07D 311/04

(52) U.S. Cl. 514/456; 549/399

(57) ABSTRACT

Methods for treating smooth muscle hyperactivity, including urinary incontinence, while avoiding concomitant liability of adverse effects associated with tolterodine and the racemic version thereof are disclosed. The methods comprise administering a therapeutically effective amount of a mono-isopropyl metabolite or a parahydroxymethyl metabolite or a parahydroxymethyl mono-isopropyl metabolite of tolterodine or racemic versions thereof or a pharmaceutically acceptable salt of either metabolite. Pharmaceutical compositions in the form of tablets and transdermal devices comprising said compounds and acceptable carriers are also disclosed.

### FIELD OF THE INVENTION

This invention relates to a compound named tolterodine and having the formula:

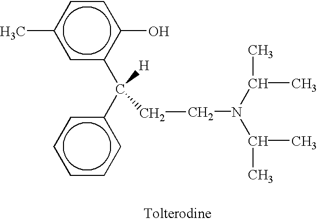

The generic name TOLTERODINE (CAS-124937-51-5; INN) refers to the R-enantiomer of the drug. In this document, the racemate of this drug is referred to as RS-tolterodine (or RS-TOLT). The R-isomer (tolterodine) is here referred to as TOLT. The chemical name of tolterodine is R(+)-N,N-diisopropyl-3-(2-hydroxy-5-methylphenyl)-3-phenylpropylamine and the chemical name of RS-TOLT is RS-N,N-diisopropyl-3-(2-hydroxy-5-methylphenyl)-3-phenylpropylamine. Des-isopropyl-tolterodine is a metabolite of TOLT and is here referred to as DES-TOLT and the racemate thereof is referred to as RS-DES-TOLT. The chemical name for RS-DES-TOLT is RS-N-Isopropyl-3-(2-hydroxy-5-methylphenyl)-3-phenylpropylamine and the chemical name of DES-TOLT is R(+)-N-isopropyl-3-(2-hydroxy-5-methylphenyl)-3-phenylpropylamine. The compound 5-hydroxymethyl-tolterodine is a metabolite of TOLT and is here referred to as 5-HM and the racemate thereof is referred to as RS-5-HM. The chemical name for RS-5-HM and 5-HM are RS-N,N-diisopropyl-3-(2-hydroxy-5-(hydroxymethyl)phenyl)-3-phenylpropylamine and R(+)-N,N-diisopropyl-3-(2-hydroxy-5-(hydroxymethyl)phenyl)-3-phenylpropylamine, respectively. The compounds DES-TOLT can undergo hepatic oxidation of the paramethyl substituent, whereby the compound 5-HM-DES-TOLT is formed. The chemical name for 5-HM-DES-TOLT is R(+)-N-Isopropyl-3-(2-hydroxy-5-(hydroxymethyl)phenyl)-3-phenylpropylamine and this compound exists in the racemic form as well as. The 5-hydroxylated compound 5-HM-DES-TOLT can undergo further oxidative metabolism and via the aldehyde, the 5-carboxylic acid metabolite is formed in the liver.

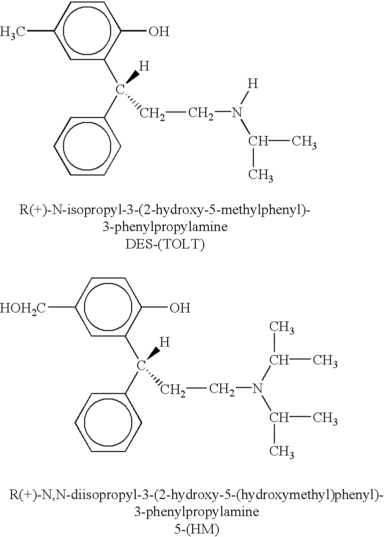

Specifically, the invention relates to processes for preparing certain metabolites of tolterodine and to methods for treating smooth muscle hyperactivity disorders using such metabolites. Smooth muscle hyperactivity disorders of the urinary bladder cause urinary disorders, including urinary incontinence and pollakiuria. Smooth muscle hyperactivity disorders of the gastrointestinal tract cause gastrointestinal disorders, including irritable bowel syndrome and diarrhea. Other smooth muscle hyperactivity disorders occur also in conjunction with asthma, urolithiasis, choledocholithiasis and cholelithiasis. The present invention describes the use of the anticholinergic compounds DES-TOLT, RS-DES-TOLT, 5-HM, RS-5-HM, 5-HM-DES-TOLT and RS-5-HM-DES-TOLT and pharmaceutical compositions containing at least one of said compounds, while avoiding side effects of the parent compounds, said parent compounds being TOLT and RS-TOLT.

### BACKGROUND OF THE INVENTION.

TOLT has been shown to reduce urinary bladder hyperactivity in patients suffering from urinary incontinence and the drug exerts a spasmolytic effect on bladder smooth muscle by inhibiting the action of acetylcholine. TOLT has selectivity for muscarinic receptors over nicotinic receptors and as a result, no blocking effects are observed at skeletal neuromuscular junctions. Like TOLT and RS-TOLT, the active metabolites thereof exert antimuscarinic activities that account for their therapeutic activities.

The compounds DES-TOLT and 5-HM have been described as major metabolites of TOLT by several investigators, such as for example Nilvebrant et al. 1997 (Antimuscarinic potency and bladder selectivity of PNU-200577, a major metabolite of tolterodine. Pharmacol Toxicol 81:169-172), Brynne et al. 1997 (Pharmacokinetics and pharmacodynamics of tolterodine in man: a new drug for the treatment of urinary bladder overactivity. Int J Clin Pharmacol Ther 35: 287-295), Andersson et al. 1998 (Biotransformation of tolterodine, a new muscarinic antagonist, in mice, rats, and dogs. Drug Metab Dispos. 26:528-535) and Postlind et al 1998 (Tolterodine, a new muscarinic receptor antagonist, is metabolized by cytochromes P450 2D6 and 3A in human liver microsomes. Drug Metab Dispos 26: 289-293). It is not known to us if the compound 5-HM-DES-TOLT, or any of the further oxidized metabolites thereof have previously been synthesized. The medicinal use of the tolterodine metabolite 5-HM has been described by Johansson et al. in U.S. Pat. No. 5,559,269 (1996) and U.S. Pat. No. 5,686,464 (1997), both with foreign application priority date Nov. 06, 1992 (SE 9203318). The medicinal use of RS-DES-TOLT or DES-TOLT or any of the paramethyl-oxidized metabolites thereof have to our knowledge not been described.

### SUMMARY OF THE INVENTION

Methods for treating smooth muscle hyperactivity, including urinary incontinence, while avoiding concomitant liability of adverse effects associated with tolterodine and the racemic version thereof are disclosed. The methods comprise administering a therapeutically effective amount of a mono-isopropyl metabolite or a parahydroxymethyl metabolite or a parahydroxymethyl mono-isopropyl metabolite of tolterodine or racemic versions thereof or a pharmaceutically acceptable salt of either metabolite. Pharmaceutical compositions in the form of tablets and transdermal devices comprising said compounds and acceptable carriers are also disclosed.

### DETAILED DESCRIPTION OF THE INVENTION

Pharmacological studies of the metabolites of tolterodine and the corresponding racemates have now been performed in comparison with tolterodine. These studies demonstrate that DES-TOLT, as well as 5-HM-DES-TOLT and the further oxidized metabolites thereof have potent antimuscarinic activities.

It has been found that TOLT and RS-TOLT cause a prolongation of the QTc-interval of the EKG. Prolongation of the QTc interval is indicative of risk for a type of fatal cardiac arrhythmias that is called torsades des Pointes, as described for terfenadine by Woosley et al. 1993 (Mechanism of the cardiotoxic actions of terfenadine. JAMA 269: 1532-1536). The risk for cardiac arrhythmias with TOLT and RS-TOLT in patients may be particularly high when one of said compounds is combined with other drugs that utilize the same metabolic enzyme as said compounds or when said compound is given to patients who are “poor metabolizers” as described by Stahl et al., 1995. However, it was surprisingly found that DES-TOLT and 5-HM as well as RS-DES-TOLT and RS-5-HM did not cause a prolongation of the QTc interval of the EKG. It is therefore concluded that DES-TOLT, 5-HM, RS-DES-TOLT, RS-5-HM, 5-HM-DES-TOLT and RS-5-HM-DES-TOLT offer anticholinergic treatment for smooth muscle hyperactivity disorders, while being devoid of electrophysiological cardiac side effects that reside in the parent compounds, said parent compounds being TOLT and RS-TOLT.

Synthesis of DES-TOLT and RS-DES-TOLT

Synthetic methods of making of DES-TOLT and RS-DES-TOLT were described by Jönsson et al. in European Patent Application 89850017.8 and are hereby incorporated by reference.

Synthesis of 5-HM and RS-5-HM

Synthetic methods of making of 5-HM and RS-5-HM were described by Johansson et al. in U.S. Pat. No. 5,559,269 and are hereby incorporated by reference.

Synthesis of 5-HM-DES-TOLT

The synthesis of 5-HM-DES-TOLT was performed by using a combination of the methods for making 5-HM and DES-TOLT as described in the above mentioned references by Jönsson et al. (European Patent Application 89850017.8) and Johansson et al. (U.S. Pat. No. 5,559,269), and as known to those skilled in the art of synthetic chemistry.

Therapeutic Doses

The magnitude of a prophylactic or therapeutic dose of a compound of the present invention in the acute or chronic management of disease will vary with the severity and nature of the condition to be treated and the route of administration. The dose and the frequency of the dosing will also vary according to the age, body weight and response of the individual patient. In general, the total daily oral dose range for DES-TOLT or 5-HM or 5-HM-DES-TOLT for the conditions described herein is from about 0.5 mg to about 100 mg in single or divided doses, preferably in divided doses or in single dose using a controlled release oral formulation. In managing the patient, the therapy should be initiated at a low dose, perhaps at 1 or 2 mg to about 10 mg orally, and may be increased up to about 50 mg depending on the patient's global response. It is further recommended that patients over 65 years and those with impaired renal or hepatic function initially receive low doses and that they be titrated based on individual response(s) and plasma drug level(s). It may be necessary to use dosages outside these ranges in some cases, particularly if the drug is administered by routes other than the oral route, as will be apparent to those skilled in the art. Further, it is noted that the clinician or treating physician will know how and when to interrupt, adjust, or terminate therapy in conjunction with individual patient response. The terms “a therapeutically effective amount” and “an amount sufficient to treat the disorder but insufficient to cause adverse effects” are encompassed by the above-described dosage amounts and dose frequency schedule.

Any suitable route of administration may be employed for providing the patient with an effective dosage of the compounds of the present invention. For example, oral, sublingual, parental (i.e. subcutaneous, intramuscular, intravenous, etc.), transdermal, vaginal, aerosol and like forms of administration may be employed. Additionally, the drug may be administered directly into the bladder, as described for oxybutynin by Massad et al. [J. Urol. 148, 595-597 (1992)] or rectally directly into the gastrointestinal canal as known in the art. Dosage forms include tablets, troches, dispersions, suspensions, solutions, capsules, suppositories, microencapsulated systems, slow-release and controlled release systems, transdermal delivery systems, and the like.

The pharmaceutical compositions of the present invention comprise of DES-TOLT, 5-HM, RS-DES-TOLT, RS-5-HM, 5-HM-DES-TOLT or RS-5-HM-DES-TOLT as the active ingredient, or a pharmaceutically acceptable salt thereof, and may also contain a pharmaceutically acceptable carrier, and optionally, other therapeutic ingredients.

The terms “pharmaceutically acceptable salts” or “a pharmaceutically acceptable salt thereof” refer to salts prepared from pharmaceutically acceptable non-toxic acids. Suitable pharmaceutically acceptable acid addition salts for the compound of the present invention include acetic, benzenesulfonic (besylate), benzoic, camphorsulfonic, citric, ethanesulfonic, fumaric, gluconic, glutamic, hydrobromic, hydrochloric, isethionic, lactic, maleic, malic, mandelic, methanesulfonic, mucic, nitric, pamoic, pathothenic, phosphoric, p-toluenesulfonic, succinic, sulfuric, tartaric, and the like. The hydrochloride is particularly preferred.

The compositions of the present invention include suspensions, solutions, elixirs or solid dosage forms. Carriers such as starches, sugars, and microcrystalline cellulose, diluents, granulating agents, lubricants, binders, disintegrating agents, and the like are suitable in the case of oral solid preparations (such as powders, capsules, and tablets), and oral solid preparations are preferred over the oral liquid preparations.

Because of their ease of administration, tablets and capsules represent the more advantageous oral dosage unit forms, in which case solid pharmaceutical carriers are employed. If desired, tablets may be coated by standard aqueous or nonaqueous techniques.

In addition to the common dosage forms set out above, the compounds of the present invention may also be administered by controlled release means and by means of various delivery devices as known by those skilled in the art. Controlled release means transdermal delivery and delivery devices include patches, ionophoretic systems and the like, as well as slow release or controlled release oral formulations.

Pharmaceutical compositions of the present invention suitable for oral administration may be presented as discrete unit dosage forms such as capsules, cachets, suppositories, or tablets, each containing a predetermined amount of the active ingredient, as a powder or granules, or as a solution or a suspension in an aqueous liquid, a non-aqueous liquid, an oil-in-water emulsion, or a water-in-oil liquid emulsion. Such compositions may be prepared by any of the methods of pharmacy, but all methods include the step of bringing into association the active ingredient with the carrier, which constitutes one or more necessary ingredients. In general, the compositions are prepared by uniformly and intimately admixing the active ingredient with liquid carriers or finely divided solid carriers or both, and then, if necessary, shaping the product into the desired presentation, just as is known for the racemic mixture. Carriers such as starches, sugars, and microcrystalline cellulose, diluents, granulating agents, lubricants, binders, disintegrating agents, and the like are suitable in the case of oral solid preparations (such as powders, capsules, and tablets), and oral solid preparations are preferred over the oral liquid preparations.

For example, a tablet may be prepared by compression or molding, optionally, with one or more accessory ingredients. Compressed tablets may be prepared by compressing in a suitable machine the active ingredient in a free-flowing form such as powder or granules, optionally mixed with a binder, lubricant, inert diluent, surface active agent or dispersing agent. Molded tablets may be made by molding, in a suitable machine, a mixture of the powdered compound moistened with an inert liquid diluent. All of the foregoing techniques are well know to persons of skill in the pharmaceutical art. Each tablet may contain from about 0.5 mg to about 25 mg of the active ingredient.

### EXAMPLE 1

### Oral Unit Dosage Formulation

Tablets:

| | | | per batch of | |
|---|---|---|---|---|
| Ingredients | per tablet | | 10,000 tablets | |
| DES-TOLT | 5 | mg | 50 | g |
| Microcrystalline cellulose | 30 | mg | 300 | g |
| Lactose | 70 | mg | 700 | g |
| Calcium stearate | 2 | mg | 20 | g |
| FD&C Blue #1 Lake | 0.03 | mg | 300 | mg |

The DES-TOLT is blended with lactose and cellulose until a uniform blend is formed. The lake is added and further blended. Finally, the calcium stearate is blended in, and the resulting mixture is compressed into tablets using a 9/32 inch (7 mm) shallow concave punch. Tablets of other strengths may be prepared by altering the ration of active ingredient to the excipients or to the final weight of the tablet.

Pharmacological Studies of Tolterodine and Metabolites Thereof

1. Ligand Binding Studies: Affinity for Muscarinic Receptors

The experiments are carried out on membranes prepared from SF9 cells infected with baculovirus to express human recombinant muscarinic receptor subtypes. After incubation with the test article and the proper radioligand (³H pirenzepine) and washing, bound radioactivity is determined with a liquid scintillation counter, using a commercial scintillation cocktail. The specific radioligand binding to a muscarinic receptor is defined as the difference between total binding and nonspecific binding determined in the presence of an excess of unlabelled ligand. IC₅₀ values (concentrations required to inhibit 50% of specific binding) are determined by non-linear regression analysis of the competition curves. These parameters are obtained by curve fitting using Sigmaplot™ software.

2. Functional Characterization of Antimuscarinic Activities on Smooth Muscle Strips

Experiments are performed using methods similar to those described by Kachur et al, 1988 (R and S enantiomers of oxybutynin: Pharmacological effects in guinea pig bladder and intestine. J Pharmacol Exp Ther 247: 867-872) and Noronha-Blob and Kachur, 1991 (Enantiomers of Oxybutynin: In vitro pharmacological characterization at M1, M2 and M3 muscarinic receptors and in vivo effects on urinary bladder contraction, mydriasis and salivary secretion in guinea pigs. J Pharmacol Exp Ther 256: 562-567). Strips of tissue (approximately 10 mm long and 1.5 mm wide) are removed from the body of the urinary bladder of male guinea pigs weighing 400-600 g. Preparations of the longitudinal smooth muscle of the colon of guinea pigs are prepared as known from the prior art (Acta Physiol Scand 64: 15-27, 1965). This method is also modified and used for the testing of the drugs on smooth muscle from the kidney, the gall bladder and the airways. The tissues are suspended in an oxygenated buffer of the following composition, in mM: NaCl 133; KCl 4.7; CaCl₂ 2.5; MgSO₄ 0.6; NaH₂PO₄ 1.3; NaHCO₃ 16.3; and glucose 7.7, or of a similar composition. The smooth muscle strips are maintained at or about 37.5 C. In each experiment up to seven strips are removed from a single animal, suspended in tissue chambers and allowed to equilibrate with the bathing solution for one hour before proceeding with the experiment. Contractions are recorded with transducers on a polygraph.

The present series of experiments focuses on the anticholinergic actions of DES-TOLT, and RS-DES-TOLT and their metabolites. In these experiments, in order to assess the viability of each tissue and to serve as a frame of reference, contractions of each strip of tissue are recorded initially in response to exposure to tissue medium in which NaCl is replaced by KCl to yield a concentration of 137.7 mM KCl in the medium. This is followed by return to the standard medium, and then by exposures to progressively in creasing concentrations of carbachol, with separate exposures to each concentration only until the peak response has been recorded. Then, leaving one strip untreated and/or one strip exposed to the test solution to serve as control tissue(s), the remaining strips each are exposed for one hour to one concentration of an antagonist. Finally, the responses to increasing concentrations of carbachol are recorded a second time.

4. Cardiac Side Effects

Male guinea pigs (450-600 g) are anesthetized with freshly prepared dialurethane sodium. The jugular vein is catheterized for iv administration of test drugs and the trachea is exposed and cannulated. Subdermal electrodes are positioned for Lead II electrocardiogram recording, monitored on a Grass Polygraph recorder, set at a paper speed of 50 mm/sec. The animals are allowed to stabilize for 30 minute after completion of surgery, and three baseline EKG recordings are then made at 10-minute intervals. The animals are then given a dose of the test compound or vehicle as an intravenous infusion over 30 min. EKG recordings are used to determine QT intervals and heart rates. To compensate for variations in heart rates, QTc intervals are calculated from QT- and RR-intervals as known to those skilled in the art. Prolongation of QTc is indicative of a prolonged action potential, caused by an inhibition of the delayed rectifier potassium channel. Prolongation of QTc is the known cause of Torsades de Pointes ventricular fibrillation by drugs such as terfenadine, astemizole and terodiline (now withdrawn from the market).

Other methods for studying cardiac side effects are also used.

### Equivalents

Those skilled in the art will recognize, or be able to ascertain, using no more than routine experimentation, many equivalents to the specific embodiments of the invention described herein. Such equivalents include numerous pharmaceutically acceptable salt forms e.g. sulfate, funarate, hydrobromide, hydrochloride, dihydrochloride, methanesulphonate, hydroxynaphthoate, or where appropriate one or other of the hydrate forms thereof, see Merck Index 11th edition (1989) items 9089, 209, 3927, 4628, 8223, 5053, 5836, 8142, 2347, 7765, 1840, 9720, 7461, 1317,4159, and 963 and references cited therein and Am. Rev. Resp. Dis. 1988, 137: (4;2/2) 32. Such equivalents also include the co-administration of at least one compound of the present invention with any other drug that is used to combat diseases in mammals, mentioned in this document. Such equivalents also include the co-administration of at least one compound of the present invention with any other compound or drug that may be used in combination with medication for urinary incontinence or other forms of smooth muscle hyperactivity. Those skilled in the art of pharmacology will realize that the pharmacologically active compounds of the present invention may also be combined with in different concentrations with cholinergically inert compounds, such as S-tolterodine or a metabolite thereof. Those skilled in the art of medicine will also realize that higher or lower doses than those indicated here may be preferred and the doses may be given more or less frequently than suggested here.

Those skilled in the art of drug metabolism will realize that 5-hydroxymethyl metabolites of TOLT or RS-TOLT can and will undergo further oxidative metabolism as described in this document. All such further oxidized metabolites, including aldehydes and the carboxylic acids are included in the present invention.

Those skilled in the art of drug metabolism will realize that DES-TOLT can and will undergo additional dealkylation, whereby a di-des-isopropyl metabolite is formed. This pharmacologically active antimuscarinic metabolite and the paramethyl-oxidized forms thereof are included in the present invention.

Those skilled in the art, will realize that smooth muscle hyperactivity disorders comprise such disorders of the urinary bladder, the gastrointestinal tract, the urinary ducts (“kidney stone pain”) the gall fluid ducts (“gall stone pains”) and the smooth muscles of the airways.

Those skilled in the art of pharmacology, will realize that the compounds of the invention, having certain pharmacological properties such as antihistaminic activity and anticholinergic activity may be useful for other indications than those listed here. Such indications include but are not limited to cardiovascular indications such as heart failure, myocardial infarction, stroke, and allergic disorders and are equivalents to the specific embodiments of the invention described herein.

Those skilled in the art know that transdermal delivery systems often contain one or more permeation enhancer(s) that dramatically may improve the transdermal absorption of a drug of this invention.

All equivalents are intended to be included in this present invention.

What is claimed is:

1. A method for treating cholinergically induced smooth muscle hyperactivity disorders, comprising the administration to a mammal in need of such treatment a therapeutically effective amount of a compound selected from the group consisting of R,S-N-isopropyl-3-(2-hydroxy-5-methylphenyl)-3-phenylpropylamine, R(+)-N-isopropyl-3-(2-hydroxy-5-methylphenyl)-3-phenylpropyl amine, RS-N-Isopropyl-3-(2-hydroxy-5-(hydroxymethyl)phenyl)-3-phenylpropylamine or R(+)-N-Isopropyl-3-(2-hydroxy-5-(hydroxymethyl)phenyl)-3-phenylpropylamine or a pharmaceutically acceptable salt thereof.

2. The method of claim 1, wherein said compound is R,S-N-isopropyl-3-(2-hydroxy-5-methylphenyl)-3-phenylpropylamine or a pharmaceutically acceptable salt thereof.

3. The method of claim 1, wherein said compound is R(+)-N-isopropyl-3-(2-hydroxy-5-methylphenyl)-3-phenylpropyl amine, or a pharmaceutically acceptable salt thereof.

4. The method of claim 1, wherein said compound is RS-N-Isopropyl-3-(2-hydroxy-5-(hydroxymethyl)phenyl)-3-phenylpropylamine or a pharmaceutically acceptable salt thereof.

5. The method of claim 1, wherein said compound is R(+)-N-Isopropyl-3-(2-hydroxy-5-(hydroxymethyl)phenyl)-3-phenylpropylamine or a pharmaceutically acceptable salt thereof.

6. A method for treating cholinergically induced smooth muscle hyperactivity disorders, comprising the administration to a mammal in need of such treatment a therapeutically effective amount of a compound selected from the group consisting of R,S-N-isopropyl-3-(2-hydroxy-5-methylphenyl)-3-phenylpropylamine, R(+)-N-isopropyl-3-(2-hydroxy-5-methylphenyl)-3-phenylpropylamine, RS-N-Isopropyl-3-(2-hydroxy-5-(hydroxymethyl)phenyl)-3-phenylpropylamine, R(+)-N-Isopropyl-3-(2-hydroxy-5-(hydroxymethyl) phenyl)-3-phenylpropylamine, RS-N,N-diisopropyl-3-(2-hydroxy-5-(hydroxymethyl)phenyl)-3-phenylpropylamine or R(+)-N,N-diisopropyl-3-( 2-hydroxy-5-(hydroxymethyl) phenyl)-3-phenylpropylamine or a pharmaceutically acceptable salt thereof, while reducing or eliminating concomitant liability of adverse side effects associated with the corresponding parent compounds, those parent compounds being RS-N,N-diisopropyl-3-(2-hydroxy-5-methylphenyl)-3-phenylpropylamine and R(+)-N,N-diisopropyl-3-(2-hydroxy-5-methylphenyl)-3-phenylpropylamine.

7. The method of claim 6, wherein said compound is R,S-N-isopropyl-3-(2-hydroxy-5-methylphenyl)-3-phenylpropylamine or a pharmaceutically acceptable salt thereof.

8. The method of claim 6, wherein said compound is R(+)-N-isopropyl-3-(2-hydroxy-5-methylphenyl)-3-phenylpropylamine or a pharmaceutically acceptable salt thereof.

9. The method of claim 6, wherein said compound RS-N-Isopropyl-3-(2-hydroxy-5-(hydroxymethyl)phenyl)-3-phenylpropylamine or a pharmaceutically acceptable salt thereof.

10. The method of claim 6, wherein said compound is R(+)-N-Isopropyl-3-(2-hydroxy-5-(hydroxymethyl)phenyl)-3-phenylpropylamine or a pharmaceutically acceptable salt thereof.

11. The method of claim 6, wherein said compound is RS-N,N-diisopropyl-3-(2-hydroxy-5-(hydroxymethyl)phenyl)-3-phenylpropylamine or a pharmaceutically acceptable salt thereof.

12. The method of claim 6, wherein said compound is R(+)-N,N-diisopropyl-3-(2-hydroxy-5-(hydroxymethyl)phenyl)-3-phenylpropylamine or a pharmaceutically acceptable salt thereof.

13. The method of claim 6, wherein said disorders are selected from the group consisting of urinary incontinence and pollakiuria.

14. The method of claim 6, wherein said compound or a pharmaceutically acceptable salt thereof is administered in a dose from about 0.5 mg to about 100 mg per day.

15. The method of claim 6, wherein said compound or a pharmaceutically acceptable salt thereof is administered by inhalation or by parenteral, transdermal, rectal, sublingual or oral administration.

16. The method of claim 6, wherein said compound or a pharmaceutically acceptable salt thereof is administered orally in the pharmaceutical unit dosage form of a tablet or capsule.

17. The pharmaceutical unit dosage form of claim 16. wherein said tablet or capsule is formulated for controlled release upon administration.
